(19) United States
(12) Patent Application Publication
(10) Pub. No.: US 2016/0242652 A1
(43) Pub. Date: Aug. 25, 2016

(54) DEVICE FOR DENTAL PLAQUE DETECTION

(71) Applicant: KONINKLIJKE PHILIPS N.V., Eindhoven (NL)

(72) Inventors: EIBERT GERJAN VAN PUTTEN, 'S-HERTOGENBOSCH (NL); BERNARDUS HENDRIKUS WILHELMUS HENDRIKS, EINDHOVEN (NL); OLAF THOMAS JOHAN ANTONIE VERMEULEN, OSS (NL); MARK THOMAS JOHNSON, ARENDONK (BE); ARNOLDUS JOHANNES MARTINUS JOZEPH RAS, MIERLO (NL)

(73) Assignee: Koninklijke Philips N.V., Eindhoven (NL)

(21) Appl. No.: 15/028,737

(22) PCT Filed: Oct. 16, 2014
(86) PCT No.: PCT/IB2014/065349

§ 371 Date: Apr. 12, 2016

(30) Foreign Application Priority Data
Oct. 16, 2013 (EP) 13188914.9

(51) Int. Cl. A61B 5/00; A61C 17/022; A61C 17/02; A61C 1/08; A61C 17/16

(52) U.S. Cl. CPC A61B 5/0088; A61B 5/4547; A61B 5/7225; A61B 5/7246; A61B 5/7282; A61B 5/74; A61C 1/088; A61C 17/16; A61C 17/0202; A61B 5/0075; A61C 17/022; A61B 2576/02; A61B 2560/0475

(57) ABSTRACT

The invention relates to a device (100) for detection of plaque (P) on teeth (T). According to a preferred embodiment, light (Iₘ) is emitted towards the teeth (T), and a part thereof as scattered from the surface of the teeth (T) and plaque (P) which may be present on the teeth (T) is recollected by a light receiving element (121). The received light (I_{sc}) is provided to a light detector (120) for generating a detection signal (x) that represents at least one property of the light (I_{sc}), which is then evaluated with respect to the presence of plaque (P) by determining at least one scattering-related quantity. The quantity may for example be a ratio or coefficient to be calculated from the spectrum of the received light (I_{sc}), or the temporal development of a speckle pattern.

### FIELD OF THE INVENTION

The invention relates to a device for dental plaque detection.

### BACKGROUND OF THE INVENTION

US 2011/314618 A1 discloses a toothbrush which is suitable for detecting and removing plaque from the surface within the oral cavity having applied thereto a fluorescent agent capable of binding to plaque on the surface. The surface is substantially simultaneously cleaned and irradiated with a light of a wavelength effective to provide a fluorescent emission when contacted with said fluorescent agent. A portion of the fluorescent emission is collected and compared to a predetermined threshold value. Depending on the outcome of the comparison, it is determined whether the device can be moved to another section. In case the device is kept in a particular section, it is determined when the device can be moved to another section.

US 2011/314618 A1 discloses that in general, two methods for detecting dental plaque are known. One method uses primary fluorescence, where the fluorescence of dental plaque or other dental material itself is monitored. The other method uses secondary fluorescence, where surfaces in the oral cavity suspected of bearing plaque are treated with a fluorescent label material, and the fluorescence emission of the label material on the oral cavity surfaces is detected to indicate the presence of dental plaque.

Other examples of documents relating to plaque detection on the basis of fluorescence include U.S. Pat. No. 6,485,300 B1 and US 2011/151409 A1.

A disadvantage of applying fluorescence for the purpose of plaque detection is that the costs involved are relatively high. Among other things, expensive optical filters are needed in order to ensure reliable detection results. As a consequence, devices for plaque detection are only suitable to be applied in a professional environment. Furthermore, as explained in the foregoing, methods using secondary fluorescence involve the application of a fluorescent label material, which is cumbersome and requires special user skills. This problem is absent in the case of methods using primary fluorescence, however, such methods are not very reliable as they yield only weak signals.

### SUMMARY OF THE INVENTION

It follows from the above that there is a need for another way of detecting plaque. The fact is that there is a need for having a device for detecting plaque which is suitable for home use, and which is capable of reliably performing its function based on another principle than fluorescence.

This object is addressed by a device for dental plaque detection according to claim 1. Preferred embodiments of the device are disclosed in the dependent claims.

According to the invention, a device for dental plaque detection is provided, which particularly comprises the following means:
light emitting means for emitting light in the direction of a tooth and thereby illuminating the tooth;
light receiving means for receiving light scattered from the illuminated tooth;
light detecting means for detecting the light received by the light receiving means and generating a detection signal representing at least one property of the detected light; and
processing means for determining at least one scattering-related quantity of the detected light on the basis of the detection signal and assessing whether plaque is present on the tooth by evaluating the at least one scattering-related quantity.

According to an insight underlying the invention, the presence of plaque on a tooth influences the way in which light is scattered from the tooth. Indeed, it has been found that it is actually possible to obtain a reliable indication of the presence of plaque on a tooth by detecting light scattered from the tooth and processing such light. Thus, in the device according to the invention, the presence of plaque can be determined based on scattering, absorption and/or speckle properties of light, which are suitable to be used for calculating a scattering-related quantity of the light. In any case, contrary to that which is known in the art, there is no need to rely upon fluorescence for finding a plaque status of teeth.

The “light emitting means” may comprise any unit or component from which light can be emitted into an adjacent space, notably into the mouth of a user. The light emitting means may typically comprise an optical component such as a lens or an optical fiber for directing and shaping the emitted light in a desired manner.

The “light receiving means” may in the simplest case just comprise an aperture through which light can pass. It may typically comprise an optical element such as a lens to provide for a desired behavior of the light detecting procedure. In another suitable embodiment, the light receiving means comprise at least one optical fiber.

The “light detecting means”, may comprise a spectrometer, a photodiode, a camera or the like for generating a detection signal to be used as a basis in the process of determining the at least one scattering-related quantity of the light scattered from an illuminated tooth, as received by the light receiving means. The “detection signal” may be any kind of signal, preferably an electrical signal such as a voltage. Moreover, the detection signal may decode its information in any appropriate way, for example as analogue or digital values.

The “processing means” may for example be realized by dedicated electronic hardware, by digital data processing hardware with associated software, or by a mixture of both, coupled to the light detecting means. The assessment with respect to the presence of plaque on the teeth may be qualitatively or quantitatively. The result of this evaluation may be further processed in any appropriate way. The user may for example be provided with a corresponding feedback signal. To that end, the device according to the invention may be equipped with indicating means for providing the user with information regarding the presence of plaque on the teeth.

The described device has the advantage that it allows for an improved treatment of dental plaque. This advantage is because the receipt of light scattered from a tooth and the determination of at least one quantity of that light allow for an automatic detection of plaque in real time. This information can be exploited in several ways, for example by providing the user with an associated feedback so that he/she can optimize the efficiency of a cleaning procedure of the tooth. By putting the invention to practice, there is no need for applying a label material for ensuring that a useful signal is obtained, contrary to what is known from the field in which fluorescence is relied upon for obtaining plaque status information.

Basically, the interaction of the emitted light with the surface of the tooth provides useful information about the presence of plaque on the tooth. In particular, this interaction comprises the scattering of the emitted light. In this context, the term “scattering” shall as usual denote a process in which light is, typically at random, reflected and/or refracted by some scattering material. The mentioned process of scattering will typically take place differently in the plaque and in the (clean) tooth such that their occurrence and intensity provides information about the presence or absence of plaque on the tooth.

In general, any property of the received light that provides the desired information about the presence of plaque can be exploited. One important example of such a property is the spectrum of the received light. Another important example is a speckle property of the light (i.e. the speckled appearance of an image of the tooth surface resulting from the interference of scattered coherent light).

In order to exploit information from the spectrum of the detected light, the light detecting means may preferably comprise a spectrometer. Additionally or alternatively, the light detecting means may comprise one or more specific spectral filters to select the relevant portion or portions of the spectrum. Preferably, a set of detectors with associated filters is provided in this case. Additionally or alternatively, the light detecting means may comprise a camera with which images can be generated. Typically, the camera will be designed and adjusted such that it can generate images of the surface of the teeth. Such images may for example be used to determine the above-mentioned speckle quantity.

The emitted light may optionally have a broadband spectrum, for example a spectrum covering wavelengths from about 350 nm to about 2,000 nm. Light with a broadband spectrum may typically be used in the above-mentioned case in which the spectrum of the received light shall be detected. Additionally or alternatively, multiple monochromatic light sources can be used to emit light at specific wavelengths that are of interest. Additionally or alternatively, the emitted light may have a high coherence, for example light from a coherent laser source. Coherent light may particularly be used for measuring speckle patterns of the light.

The processing means may be adapted to compare an actual value of the at least one scattering-related quantity of the detected light to a reference value of the scattering-related quantity. The reference value may for example correspond to average values determined in previous experiments.

According to another option, the determination of the scattering-related quantity of the detected light may particularly comprise the evaluation of a temporal development of this quantity. This approach exploits the fact that dental plaque may show a characteristic temporal behavior of its optical properties under certain circumstances, for example when its environment changes from dry to humid or vice versa. The temporal development may for example be found by determining a temporal correlation of the respective property over the observed period of time.

The light receiving means may optionally comprise a first inlet for light and a second inlet for light such that emitted light can be collected (after it has scattered from the surface of the teeth) at two different positions. A comparison of these fractions of received light may in some situations provide information about the presence of plaque. This may particularly be the case if the first inlet and the second inlet for light are disposed at different distances from the light emitting means. The spectrum of light received at a short distance from the light emitting means and the spectrum of light received at a larger distance from the light emitting means may for example be different due to the different path lengths these components of the emitted light have travelled through plaque. Light that has traveled over a larger distance has typically also travelled deeper through the material.

The light that is emitted from the light emitting means may in general originate from any appropriate source, including a light source external to the device (and even natural light). However, in a preferred embodiment, the device comprises a light source for generating the emitted light. The light source may for example be a LED or a laser incorporated into the device. In case the device is a toothbrush, the light source may optionally be disposed in the head of the toothbrush. Because the head of a toothbrush, particularly an electrical toothbrush, is usually a disposable, it is however practical to have the light source (and typically also the light detecting means) in the handle of the toothbrush, using for example optical fibers to transport the light. On the other hand, is may be desirable to have the light emitting means and/or the light receiving means arranged in the head of the toothbrush such that information about plaque immediately from a position that is currently treated can be obtained.

For the sake of completeness, it is noted that as usual, a “toothbrush” shall denote a device for manually or electrically cleaning the teeth (of a human user or of an animal). A toothbrush typically comprises a handle that is connected via a neck to a head that carries means for cleaning the teeth, for example a brush with bristles. An electrical toothbrush may additionally comprise elements such as a battery and a motor for moving the brush.

In general, the device according to the invention may be equipped with teeth cleaning means for subjecting the teeth to be evaluated as to their plaque status to a cleaning action. For example, the device may comprise an injector for injecting a stream of fluid such as a stream of gas (e.g. air) and/or liquid (e.g. water). By the injection of an appropriate fluid, the process of detecting and/or treating plaque can be assisted. A stream of water can for example remove tooth paste that might impair the illumination of the teeth by the emitted light, or a stream of air can expose plaque to a dry environment such that a particular temporal behavior is triggered.

The aforementioned injector may preferably comprise a flow channel through which the stream of fluid is led, wherein the flow channel and the light emitting means may be positioned with respect to each other in such a way that the injected stream of fluid and the emitted light may leave the device in substantially the same direction. The stream of fluid can then establish reproducible conditions for the light related measurements.

In general, parameters of a cleaning procedure such as a mechanical cleaning intensity, the delivery of a cleaning agent or the like may automatically be controlled/adapted by suitable controlling means on the basis of the assessment of the plaque status of the teeth.

The light emitting means and/or the light receiving means may preferably comprise a light guide such as an optical fiber. This allows to emit and/or receive light at an optimal position that can be chosen independently of the position of light generation and/or detection, respectively.

In another preferred embodiment, the device according to the invention may be a toothbrush as mentioned earlier, wherein the light emitting means may be located in a bristle of the toothbrush. Such light emitting means may for example be realized by a fiber of the aforementioned kind. Light can thus be emitted immediately onto the surface of the teeth.

The described operation of the device according to the invention will typically be realized with the help of a computing device, e.g. a microprocessor or an FPGA in the device. Accordingly, the invention further includes a computer program product which provides the desired functionality when executed on a computing device.

Further, the invention includes a data carrier, for example a floppy disk, a hard disk, an EPROM, a compact disc (CD-ROM), a digital versatile disc (DVD), or a USB stick which stores the computer program product in a machine readable form and which operates the various means of the device according to the invention for carrying out their function in a process of dental plaque detection when the program stored on the data carrier is executed on a computing device. The data carrier may particularly be suited for storing the program of the computing device mentioned in the previous paragraph.

Nowadays, such software is often offered on the Internet or a company Intranet for download, hence the invention also includes transmitting the computer product according to the invention over a local or wide area network.

### BRIEF DESCRIPTION OF THE DRAWINGS

The various aspects of the invention will be apparent from and elucidated with reference to the embodiments described hereinafter.

In the drawings:

FIG. 1 schematically illustrates the application of a toothbrush according to a first embodiment of the invention;

FIG. 2 schematically illustrates an injector with a hollow core through which a stream of fluid can be injected;

FIG. 3 schematically illustrates an injector with a channel around a core through which a stream of fluid can be injected;

FIG. 4 schematically illustrates the collection of scattered light at different distances from a light emitting element;

FIG. 5 shows measured reflection spectra from a tooth partly covered with plaque (dashed lines) and a tooth partly cleaned (solid line), wherein the top diagram shows spectra measured with a short distance probe, the middle diagram shows spectra measured with a long distance probe, and the bottom diagram shows the ratio between these two spectra;

FIG. 6 shows a histogram of the slopes in a specific part of the spectra for 12 measurements on clean teeth (right bars “N”) and for 12 measurements on teeth covered with plaque (left bars “P”);

FIG. 7 illustrates plaque detection based on speckle detection; and

FIG. 8 is a diagram showing the measured correlation over time of a speckle pattern of light scattered from a clean tooth (“N”) and from a plaque covered tooth (“P”).

Like reference numbers or numbers differing by integer multiples of 100 refer in the Figures to identical or similar components.

### DETAILED DESCRIPTION OF EMBODIMENTS

Dental plaque is defined clinically as structured, resilient, yellow greyish substance that adheres tenaciously to the intraoral hard surfaces, including removable and fixed restorations. It is an oral bio-film characterized by its organized structure consisting of a multitude of bacteria and fluid-filled channels, particularly of bacteria in a matrix of salivary glycoproteins and extracellular polysaccharides.

Based on its position on tooth surface, dental plaque may be classified into supragingival plaque or subgingival plaque. The maturation of oral plaque is very variable, depending on location in the mouth, age, time, oral environment and other factors. Despite this variability, oral plaque develops according to reproducible patterns.

The invention is generally related to oral healthcare, in particular to a technology to support the hygiene and health of teeth and to help users to clean their teeth from plaque.

The aforementioned objective may particularly be achieved by informing users if they are indeed removing plaque from their teeth and if they have fully removed the plaque, providing both reassurance and coaching them into good habits. Advantageously, such information is provided in real time during brushing. For example, it might be useful if a toothbrush gives the users a signal (for example an audible signal) when the position at which they are brushing is clean, so they can move to the next tooth. This may reduce their brushing time, but will also lead to a better, more conscious brushing routine.

In view of the above, it is proposed to illuminate teeth with light; by measuring the light, or part of the light, which scatters from the teeth, it is then possible to determine the presence or absence of plaque. This provides a way to detect plaque in real-time during the brushing routine. In particular, an embodiment of this approach may consist of a device comprising:
a light source to illuminate the surface of the tooth;
an optical detector to detect the light that returns from the tooth, which can for example be a sensor with filter, a spectrometer, a camera or a combination; and
a processor for analyzing the detected light, wherein the presence of plaque is determined based on scattering, absorption and/or speckle properties of light.

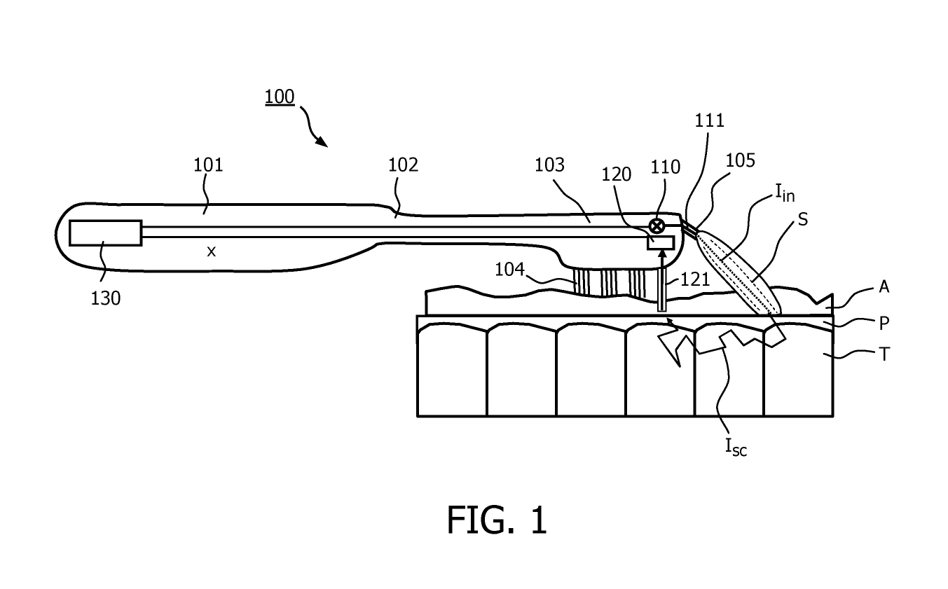

FIG. 1 schematically illustrates the application of a toothbrush 100 that is designed according to the above general principles. The toothbrush 100 comprises a handle 101 where it can be held by a user and a head 103 with a brush comprising bristles 104, wherein the handle 101 and the head 103 are connected by an elongated neck portion 102. The toothbrush is illustrated during its application to teeth T covered by plaque P and during the simultaneous application of toothpaste A.

In order to provide for the desired detection and monitoring of plaque, the toothbrush 100 further comprises the following components:
A light source 110 for controllably generating light, which may for example be an LED or a laser.
An outlet element 105 via which the shown light Iᵢₙ generated by the light source 110 and a stream S of air and/or water can be emitted towards the teeth T. To this end, the outlet element 105 comprises a light emitting element, here in the form of a light guiding element 111, for example an optical fiber that guides light from the light source 110 to the end of the outlet element 105. Moreover, a flow channel is provided in the outlet element 105 through which a stream S of air/water can be injected into the mouth of the user.
A light receiving element 121, here realized by a light fiber that is disposed among the bristles 104 of the toothbrush 100. The light receiving element 121 receives light I_{sc} that has been scattered on the surface of the teeth T and/or by the plaque P and leads this to a light detector.
The aforementioned light detector 120, which may for example be a photodiode or a camera. The light detector 120 generates a detection signal x that indicates at a property of the received light I_{sc}.
An evaluation unit 130, for example a microprocessor integrated into the handle 101 of the toothbrush 100, that is connected to the light detector 120 and to the light source 110 for controlling them and for receiving (and evaluating) the detection signal x. The evaluation of the signal x involves a calculation of a suitable scattering-related quantity as will be explained later.

The described setup allows for the emission of light Iᵢₙ onto the surface of teeth T, i.e. onto the plaque P if such is present. The light will then be scattered and a part of it will be taken up by the light receiving element 121 and forwarded to the detector 120 for evaluation.

During practical tests, it has been found that the procedure works best in the absence of tooth paste. It is therefore preferred that the detection of plaque P is done together with a method to (temporarily) remove the tooth paste A. This can for example be achieved by built-in air floss, water jets, and/or air jets, which may be emitted through the described outlet element 105.

Another option would be to employ an optical fiber to deliver and detect light directly from the surface of the tooth T. This optical fiber could be brought in direct contact with the surface to ensure that no or little tooth paste A is present.

A further preferred embodiment is to direct an air/water jet to the tip of the aforementioned fiber. The air/water could be directed with e.g. a hollow core that is positioned parallel along the length of the optical fiber.

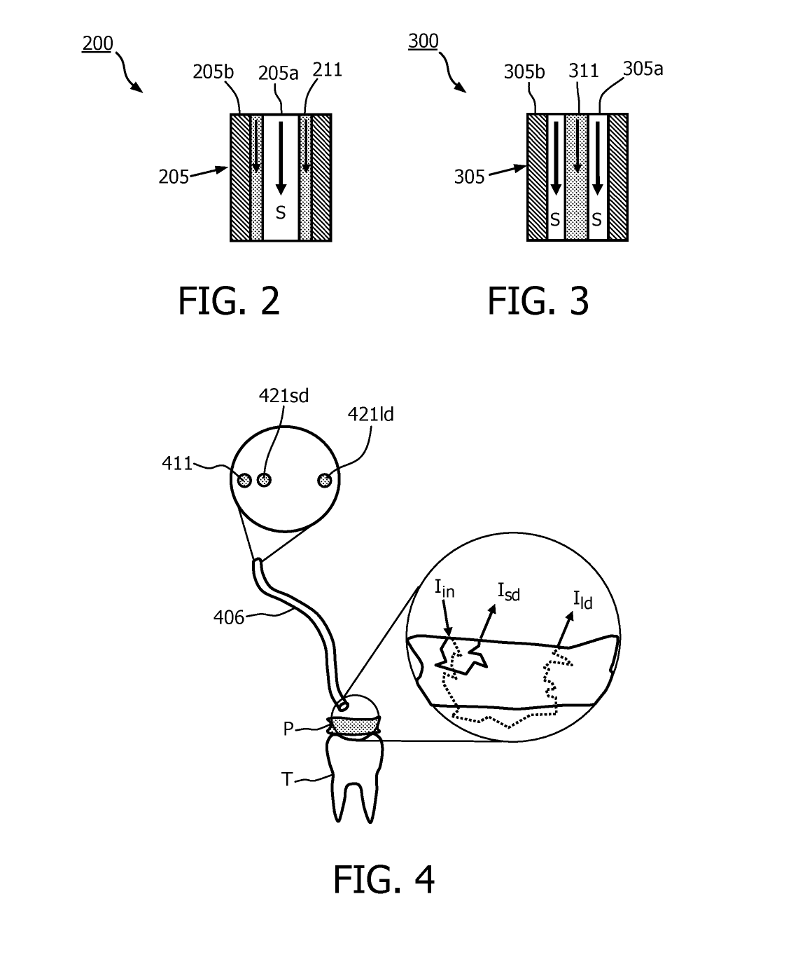

One possible non-limiting embodiment of this is shown in FIG. 2 which relates to a toothbrush 200. A combined outlet element 205 for light and a stream of air/water comprises a hollow core 205a through which a stream S of air or water can flow and a light guiding part 211 around it.

FIG. 3 relates to a toothbrush 300 and shows an alternative embodiment in which light is guided through a fiber at the core 311 and where a stream S of air or water flows in a partially hollow area 305a surrounding the core 311.

The outlet elements 205, 305 of FIGS. 2 and 3 further comprise an outer mantle 205b, 305b. The outlet element 105 of FIG. 1 may optionally be designed in this way, too.

FIG. 4 illustrates an alternative embodiment of a toothbrush 400 (only the relevant components are shown) which uses an optical probe 406 with multiple fibers to illuminate the teeth T and detect scattered light at multiple distances from the illumination point. The spectrum of the light changes as it propagates through the tooth T. These spectral changes due to absorption and scattering depend, among others, on the absence or presence of plaque P.

The optical probe 406 contains a light emitting element 411, i.e. one fiber through which light from a light source (not shown) is guided and finally emitted. Furthermore, the probe 406 contains a “short distance light receiving element” 421sd and a “long distance light receiving element” 421ld, both of them also being realized by a single fiber that is arranged a short distance and long distance away from the light emitting element 411, respectively. The light received by these two receiving elements 421sd, 421ld can be separately detected, for example because the receiving elements 421sd, 421ld are coupled to distinct light detectors such as spectrometers (not shown) or other simpler detectors (e.g. two or three photodiodes with spectral filters, depending on the measurement scheme).

The short distance light receiving element 421sd can collect light I_{sd} that has been emitted by the light emitting element 411 and scattered through the teeth T and the plaque P, but only for a short distance, while the long distance light receiving element 421ld collects light I_{ld} that has been scattered over a longer distance. Accordingly, the properties of these two fractions of received light are different.

During usage, light Iᵢₙ from a broadband light source is injected through the fiber of the light emitting element 411. The light receiving fibers 421sd, 421ld are connected to spectrometers which enable a measurement of the spectrum or parts of the spectrum.

It is preferred for both the light emitting element 411 and the light receiving fibers 421sd, 421ld to be arranged for directly contacting the surface of the teeth T, in order to minimize exterior influences. Within the framework of the invention, it is possible to use three optical fibers 411, 421sd, 421ld for emitting light and receiving light, but other possibilities are feasible as well. For example, only two fibers can be used, wherein it may be so that one fiber serves for emitting light, and another fiber serves for receiving light, or that one fiber serves for both emitting light and receiving light, and another fiber serves for only receiving light, for example. It is also possible to use a single fiber for performing both functions of emitting light and receiving light, as mentioned. In a case of a fiber having a combined function, it is appropriate to use a splitter at the side of the fiber where the light source and the detector are present. In any case, when at least one optical fiber is used, it is a practical option to have the fiber arranged in the brush of the toothbrush 400.

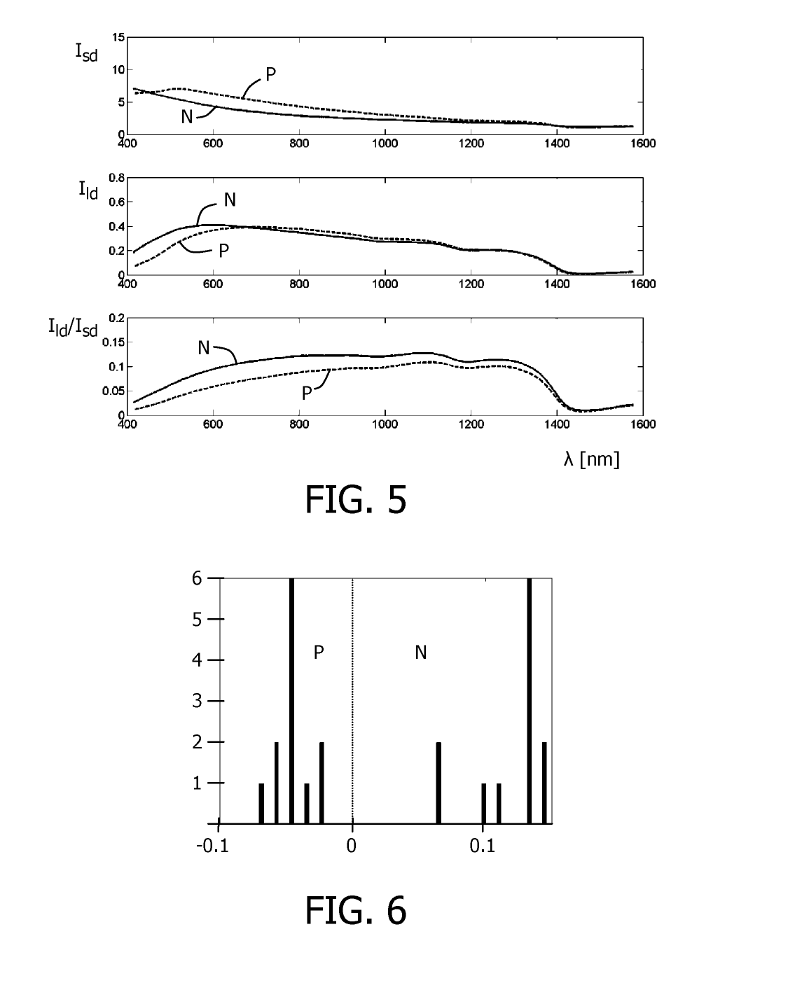

FIG. 5 shows a typical reflection spectrum from a part of a tooth T covered with plaque P (dashed lines, symbol “P”) and a part of a tooth T that was clean (solid lines, symbol “N”). The top graph shows the spectra (as normalized intensity I_{sd}) measured with the short distance light receiving element 421sd, the middle graph the spectra from the long distance light receiving element 421ld (as normalized intensity I_{ld}), and the bottom graph displays the ratio between the two spectra (I_{ld}/I_{sd}). There are clear differences between these spectra, allowing for the detection of plaque P.

In the following, three exemplary methods to extract the presence of plaque P from the spectra will be described in more detail.
1. In a first approach, the ratio between the two spectra (I_{ld}/I_{sd}) is determined as illustrated in the bottom graph of FIG. 5. An advantage of using both spectra is that the influence of noise factors like detection errors, influence of environmental light, etc., which can be assumed to be the same at both distances from the light emitting element 411, can be averaged out, so that the ratio can be used as a reliable indicator of the plaque status of the teeth T. It can be assumed that when plaque P is present on a tooth T, absorption effects are relatively small, so that such effects do not hinder practical application of the ratio in the specific context of plaque detection.

Preferably, the ratio is determined at a wavelength where the difference between a ratio associated with a clean tooth T and a ratio associated with a tooth T covered with plaque P is relatively large, e.g. 700 nm. By comparing an actual value of the ratio to the value of the ratio associated with a clean tooth T, for example, and assessing whether the first ratio is significantly reduced with respect to the second ratio, or not, it can be determined whether plaque P is present on the tooth T under investigation, or not.
2. In a second approach, the measured spectra are fitted to a scattering model which is especially suitable for making a distinction between scattering effects and absorption effects (see R. Nachabe et al., “Diagnosis of breast cancer using diffuse optical spectroscopy from 500 to 1600 nm: comparison of classification methods”, J. Biomedical Optics 16(8), 087010 (August 2011)). As described in this reference, the reduced scattering coefficient μ′ₛ(λ) as a function of the wavelength is modeled by

$${μ}_{s}^{′} ( λ ) = α [ {ρ ( \frac{λ}{{λ}_{0}} )}^{- b} + ( 1 - ρ ) {( \frac{λ}{{λ}_{0}} )}^{- 4} ]$$

where corresponds to a wavelength normalization value, α is the reduced scattering amplitude at , the Mie scattering slope is b, and ρ denotes the Mie-to-total reduced scattering assuming Mie and Rayleigh scattering as the two types of scattering in tissue, wherein it is noted that Mie scattering is associated with relatively large particles and Rayleigh scattering with relatively small particles. The following table shows extracted parameters using this scattering model for four different “short distance” measurements on two teeth T, both with and without plaque P, wherein λ==800 nm, and wherein μ′ₛ(λ)=α as a result of choosing the value of the wavelength such as to equal the normalization value. For the sake of completeness, it is noted that it is also possible to choose the value of the wavelength such as to deviate from the normalization value. Furthermore, another value than 800 nm may be chosen in respect of the normalization value. However, in view of the fact that the extent to which light is scattered from a surface is also dependent on the relation of the wavelength of the light with respect to the size of particles as present on the surface, 800 nm appears to be a suitable value in respect of plaque detection.

| Measurement | μ′ₛ (λ) = α (cm⁻¹) | B | ρ |
|---|---|---|---|
| tooth 1 no plaque | 16.21 | 0.88 | 1.00 |
| tooth 1 with plaque | 26.58 | 0.68 | 0.79 |
| tooth 2 no plaque | 18.38 | 0.33 | 0.80 |
| tooth 2 with plaque | 27.47 | 0.87 | 0.83 |

The results suggest the reduced scattering coefficient at a wavelength of 800 nm as a possible plaque differentiator. A tooth surface containing plaque P is rougher compared to a tooth surface without plaque P, resulting in higher scattering for a tooth T with plaque P, and the reduced scattering coefficient is higher for teeth T with plaque P compared to teeth T without plaque P. Hence, when an actual value of the reduced scattering coefficient is compared to a predetermined value of the reduced scattering coefficient associated with the absence of plaque P on the tooth T, and it is found that the first value is higher than the second value, it is concluded that plaque P is present on the tooth T. An advantage of the second approach is that only a single detector position is needed, and that very accurate results are obtained as the influence of absorption is removed.

In fact, it can be said that α provides an indication of the extent to which light is scattered from the tooth surface, b provides an indication of the Mie component of the scattered light, and ρ provides an indication of the Rayleigh component of the scattered light. The various values are determined by measuring spectra of the scattered light. It follows from the foregoing that the value α is suitable to be used as an indicator of the presence of plaque. According to a more sophisticated option, it is also possible to use the value b and/or the value ρ.
3. In a third approach, scattering at two wavelengths is evaluated. As can be seen in the top graph of FIG. 5, the slope between approximately 400 nm and 550 nm is different in the presence of plaque P. Therefore, a test was performed in which the following ratio S was calculated

$$S = \frac{I ( {λ}_{1} ) - I ( {λ}_{2} )}{I ( {λ}_{1} ) + I ( {λ}_{2} )}$$

for 24 different measurements, wherein 434 nm was taken as and 537 nm was taken as λ₂. A histogram of the results is shown in FIG. 6. All of the measurements on teeth T that contained plaque P (symbol “P”) have a negative ratio, while on clean teeth T (symbol “N”) the ratio is positive. Hence, it was concluded that the ratio is suitable to be used as a plaque differentiator, even though the influence of absorption is not removed as in the second approach. The algorithm is relatively simple, which may be an advantage in view of home use and costs of the device. In general, it is advantageous if λ₂ is chosen such as to be larger than λ₁ on the slope as mentioned.

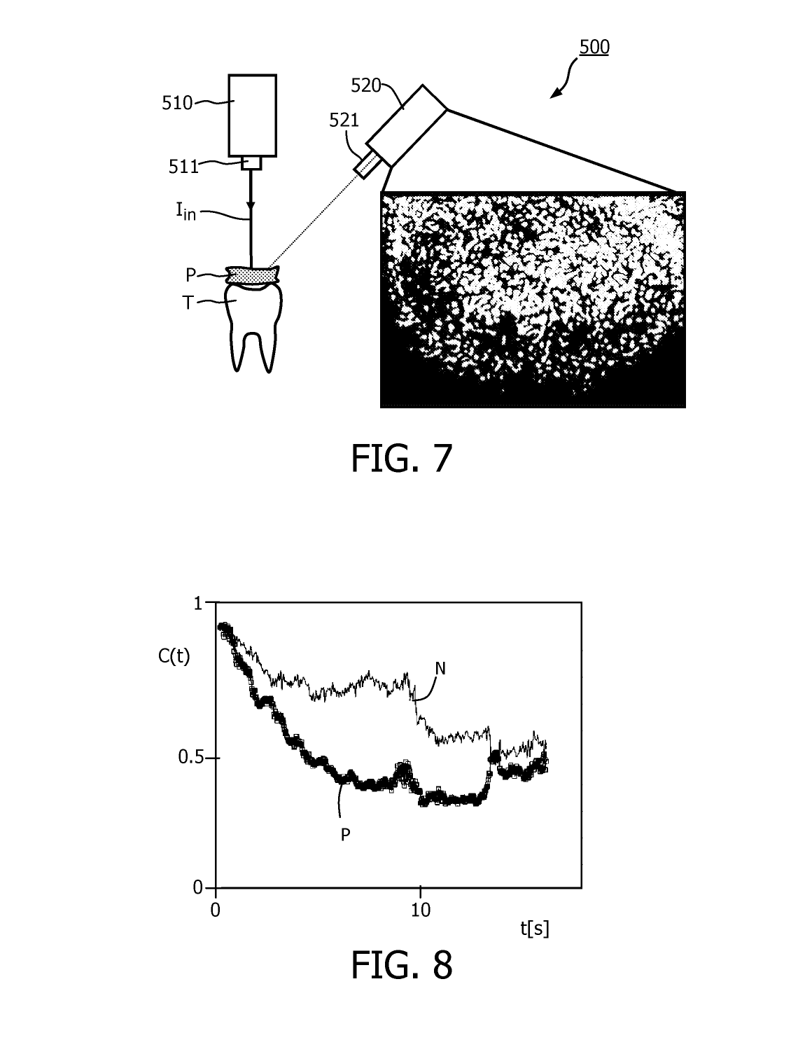

FIG. 7 schematically illustrates an alternative embodiment in which plaque detection is based on speckle correlation. Again, only the relevant components of the corresponding toothbrush 500 are shown. These comprise a coherent light source 510, for example a laser, from which light Iᵢₙ is emitted by a some light emitting element 511 towards plaque P on a tooth T. Light that has been reflected is collected by a light collecting element 521, e.g. a lens system, and guided to a (digital) camera 520 where an image of the surface is generated.

A coherent beam of light Iᵢₙ that is emitted by the laser 510 illuminates the tooth T. The camera 520 images a speckle pattern at the tooth surface, which could be covered with plaque P. As time progresses, the microscopic structure of the plaque layer P changes due to various reasons, such as water that leaks out of the layer. The change in the plaque layer leads to a measurable decorrelation of the speckle pattern. If there is no plaque P, the speckle pattern is more stable in time.

A correlation C(t) can be calculated as

$$C ( t ) = \frac{{Σ}_{x , y} [ I ( x , y , 0 ) - \overline{I ( x , y , 0 )} ] [ I ( x , y , t ) - \overline{I ( x , y , t )} ]}{{Σ}_{x , y} [ I ( x , y , 0 ) - \overline{I ( x , y , 0 )} ] [ I ( x , y , 0 + δ ) - \overline{I ( x , y , 0 + δ )} ]} ,$$

where I(x,y,t) is the measured intensity image at time t, the two sums run over the x and y pixels of the region of interest, and where the top bar denotes a spatial average value of the image I(x,y,t). The region of interest ideally covers more than one resolvable speckle. The correlation C(t) is normalized using two images: the reference image I(x,y,0) and another image, I(x,y,0+δ), which is taken very briefly after the first image such that the speckle pattern is not yet significantly decorrelated. In this way, any noise in the images averages out correctly in the normalization.

FIG. 8 shows the measured correlation of a speckle pattern of light scattered from a clean tooth (symbol “N”) and from a plaque covered tooth (symbol “P”). Just before the measurement the tooth was taken out of a watery environment and placed in a dry cup. The speckle pattern on the plaque covered surface clearly decorrelates more and faster than the speckle pattern on the clean part of the tooth. Hence, an indication of the plaque status can be obtained by comparing the value of the correlation C(t) to a predetermined value of the correlation C(t) associated with the absence of plaque on the tooth, wherein it is concluded that plaque is present if it is found that the value of the correlation C(t) is reduced with respect to the predetermined value.

The measurement works best when the tooth is in a more or less dry environment. In practice this could be achieved by using a toothbrush with a built in air floss system. The air floss could then blow the tooth dry just before the measurement starts.

Also to prevent any high frequency disturbance of the measurement due to the movement of the toothbrush, it is preferred to disable the brushing during the measurement.

In summary, various embodiments of a device for detection of plaque P on teeth T have been described. According to a preferred embodiment, light Iᵢₙ is emitted towards the teeth T, and a part thereof as scattered from the surface of the teeth T and plaque P which may be present on the teeth T is recollected by a light receiving element 121, 421sd, 521ld, 521. The received light I_{sc}, I_{sd}, I_{ld} is provided to a light detector 120, 520 for generating a detection signal x that represents at least one property of the light I_{sc}, I_{sd}, I_{ld}, which is then evaluated with respect to the presence of plaque P by determining at least one scattering-related quantity. The quantity may for example be a ratio or coefficient to be calculated from the spectrum of the received light I_{sc}, I_{sd}, I_{ld}, or the temporal development of a speckle pattern.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items/means recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1-5. (canceled)

6. Device for dental plaque detection, comprising:
light emitting means for emitting light in the direction of a tooth (T) and thereby illuminating the tooth (T);
light receiving means for receiving light scattered from the illuminated tooth (T) at two different distances from the light emitting means;
light detecting means for detecting the light received by the light receiving means and generating a detection signal (x) representing at least one property of the detected light; and
processing means for determining a ratio R of the intensity of the detected light (I_{sd}, I_{ld}) at the two different distances, particularly a ratio R which is defined as
$$R = \frac{{I}_{ld} ( λ )}{{I}_{sd} ( λ )}$$
where I_{ld}(λ) represents the intensity of the light at a wavelength λ detected at a relatively long distance from the light emitting means, and I_{sd}(λ) represents the intensity of the light at the same wavelength λ detected at a relatively short distance from the light emitting means.

7. Device according to claim 6, wherein the processing means are adapted to compare the value of the ratio R to a predetermined value of the ratio R associated with the absence of plaque (P) on the tooth (T), and to determine that plaque (P) is present if it is found that the value of the ratio R is reduced with respect to the predetermined value.

8. Device for dental plaque detection, comprising:
light emitting means for emitting light in a spectrum of wavelengths in the direction of a tooth (T) and thereby illuminating the tooth (T);
light receiving means for receiving light scattered from the illuminated tooth (T);
light detecting means for detecting the light received by the light receiving means and generating a detection signal (x) representing at least one property of the detected light; and
processing means adapted to determine a reduced scattering coefficient μ′ₛ(λ) of the detected light as a function of the wavelength λ of the light, particularly a reduced scattering coefficient which is defined as
$${μ}_{s}^{′} ( λ ) = α [ {ρ ( \frac{λ}{{λ}_{0}} )}^{- b} + ( 1 - ρ ) {( \frac{λ}{{λ}_{0}} )}^{- 4} ]$$
where λ₀ represents a wavelength normalization value, α represents the reduced scattering amplitude at λ₀, b represents the Mie scattering slope, and ρ represents the Mie-to-total reduced scattering fraction.

9. Device according to claim 8, wherein the processing means are adapted to compare the value of the reduced scattering coefficient μ′ₛ(λ) to a predetermined value of the reduced scattering coefficient μ′ₛ(λ) associated with the absence of plaque (P) on the tooth (T), and to determine that plaque (P) is present if it is found that the value of the reduced scattering coefficient μ′ₛ(λ) is higher than the predetermined value.

10. Device for dental plaque detection, comprising:
light emitting means for emitting light in a spectrum of wavelengths in the direction of a tooth (T) and thereby illuminating the tooth (T);
light receiving means for receiving light scattered from the illuminated tooth (T);
light detecting means for detecting the light received by the light receiving means and generating a detection signal (x) representing at least one property of the detected light; and
processing means are adapted to determine a ratio S of the intensity of the detected light at two different wavelengths, particularly a ratio S which is defined as
$$S = \frac{I ( {λ}_{1} ) - I ( {λ}_{2} )}{I ( {λ}_{1} ) + I ( {λ}_{2} )}$$
where I represents the normalized intensity, λ₁ represents a first wavelength, and λ₂ represents a second wavelength.

11. Device according to claim 10, wherein the second wavelength is larger than the first wavelength.

12. Device according to claim 10, wherein the values of the two different wavelengths are chosen in order to have a negative value of the ratio S when plaque (P) is present on the tooth (T) and a positive value of the ratio S when plaque (P) is absent.

13. (canceled)

14. Device for dental plaque detection, comprising:
light emitting means for emitting light in the direction of a tooth (T) and thereby illuminating the tooth (T);
light receiving means for receiving light scattered from the illuminated tooth (T);
light detecting means for detecting the light received by the light receiving means are adapted to image a speckle pattern at the tooth surface at two different instances; and
processing means adapted to determine a correlation C(t) of the two different speckle patterns, wherein the correlation C(t) of the two different speckle patterns is calculated as
$$C ( t ) = \frac{{Σ}_{x , y} [ I ( x , y , 0 ) - \overline{I ( x , y , 0 )} ] [ I ( x , y , t ) - \overline{I ( x , y , t )} ]}{{Σ}_{x , y} [ I ( x , y , 0 ) - \overline{I ( x , y , 0 )} ] [ I ( x , y , 0 + δ ) - \overline{I ( x , y , 0 + δ )} ]}$$
where x,y represent the pixels of the region of interest, I(x,y,0) represents a measured intensity image of the region of interest at time 0, which is a reference image, I(x,y,t) represents a measured intensity image of the region of interest at time t>0, I(x,y,0+δ) represents a measured intensity image of the region of interest which is taken immediately after the reference image, and the top bars indicate spatial average values of the images.

15. Device according to claim 14, wherein the processing means are adapted to compare the value of the correlation C(t) to a predetermined value of the correlation C(t) associated with the absence of plaque (P) on the tooth (T), and to determine that plaque (P) is present if it is found that the value of the correlation C(t) is reduced with respect to the predetermined value.

16-17. (canceled)

18. Device according to claim 6, being an electrical toothbrush adapted to clean teeth (T) under the influence of at least one of a stream (S) of fluid or a brushing action, wherein the device further comprises controlling means which are adapted to deactivate the teeth cleaning means during activation of the light detecting means.

19-20. (canceled)

21. Device according to claim 6, further comprising indicating means for providing a user of the device with information regarding the presence of plaque (P) on the tooth (T).
